(19) United States
(12) Patent Application Publication
(10) Pub. No.: US 2024/0102897 A1
(43) Pub. Date: Mar. 28, 2024

(54) FECAL GRINDER ASSEMBLIES, FECAL SYRINGE ASSEMBLIES, FECAL MIXERS, AND METHODS OF USING SAME

(71) Applicant: IDEXX Laboratories, Inc., Westbrook, ME (US)

(72) Inventors: Mark Dumont, Scarborough, ME (US); Chase Leffers, Boston, MA (US); Anne Leavitt, Bath, ME (US)

(73) Assignee: IDEXX Laboratories, Inc., Westbrook, ME (US)

(21) Appl. No.: 18/458,502

(22) Filed: Aug. 30, 2023

Related U.S. Application Data
(60) Provisional application No. 63/409,392, filed on Sep. 23, 2022.

(51) Int. Cl. G01N 1/28; B01D 29/33; B01D 29/58; B01F 27/112; B01F 35/00; G01N 1/38

(52) U.S. Cl. CPC G01N 1/286; B01D 29/33; B01D 29/58; B01F 27/112; B01F 35/187; G01N 1/38; B01F 2101/23

(57) ABSTRACT

A fecal grinder assembly includes a container defining an open interior volume and a plurality of container teeth extending from a bottom wall of the container and within the open interior volume, a grinder slidably positioned within the open interior volume of the container, the grinder including a base and a key extending from the base in a first direction and a plurality of grinder teeth extending from the base in a second direction opposite the first direction, and a collection member including a filter portion and a collection body extending from the filter portion, a keyway extends through the filter and the body for receiving the key of the grinder when the collection member is positioned on the grinder and within the open interior volume of the container.

### CROSS-REFERENCE TO RELATED APPLICATION

The present application claims priority to and the benefit of U.S. Provisional Patent Application 63/409,392 filed on Sep. 23, 2022, the contents of which is hereby incorporated by reference in its entirety.

### TECHNICAL FIELD

The present specification generally relates to sample collection apparatuses and methods for using the same, and, more specifically, to fecal collection apparatuses for collecting a fecal sample and preparing the fecal sample for testing.

### BACKGROUND

To identify eggs, ova, and/or perform antigen testing on a fecal sample, a user may collect a fecal sample for shipment to a testing facility. This requires the testing facility to handle the fecal sample to be tested, which may result in contamination or sample spoilage. In addition, conventional fecal testing devices such as, for example, fecal floats, process a fecal sample using a centrifuge to isolate parasites, parasite eggs, and ova from the fecal sample. However, this centrifuge technique requires an excessive amount of time to prepare the fecal sample for processing, testing, and examination while one must wait for the parasites, parasite eggs, and ova to float to a top of a container in which the fecal sample is stored.

Accordingly, a need exists for alternative sample collection apparatuses for collecting a fecal sample and permitting testing of the fecal sample while reducing handling of the fecal sample during testing, as well as reduce the amount of processing time of the fecal sample prior to examination.

### SUMMARY

In one embodiment, a fecal grinder assembly includes: a container defining an open interior volume and a plurality of container teeth extending from a bottom wall of the container and within the open interior volume; a grinder slidably positioned within the open interior volume of the container, the grinder comprising a base and a key extending from the base in a first direction and a plurality of grinder teeth extending from the base in a second direction opposite the first direction; and a collection member comprising a filter portion and a collection body extending from the filter portion, a keyway extends through the filter portion and the collection body for receiving the key of the grinder when the collection member is positioned on the grinder and within the open interior volume of the container.

In another embodiment, a fecal syringe assembly includes: a syringe body comprising a dispensing end, a receiving end opposite the dispensing end, and a nozzle formed at the dispensing end; a plunger partially insertable into the syringe body through the receiving end, the plunger comprising a collection portion and a gripping portion opposite the collection portion, the gripping portion comprising an outer diameter greater than an inner diameter of the receiving end of the syringe body; and a filter member provided at one of the nozzle or within the syringe body proximate the collection portion of the plunger when the plunger is inserted into the receiving end of the syringe body.

In yet another embodiment, a fecal mixer includes: a container comprising a dispensing end, a receiving end opposite the dispensing end, and a nozzle formed at the dispensing end; a filter member positioned within the container proximate the dispensing end; a plunger insertable into the container through the receiving end, the plunger comprising a sealing member encircling a lower wall portion of the plunger; and a mixer comprising a shaft extending through the plunger, a mixing blade provided at a first end of the shaft, the mixing blade positioned between the filter member and the plunger when the plunger is inserted into the container.

These and additional features provided by the embodiments described herein will be more fully understood in view of the following detailed description, in conjunction with the drawings.

### BRIEF DESCRIPTION OF THE DRAWINGS

The embodiments set forth in the drawings are illustrative and exemplary in nature and not intended to limit the subject matter defined by the claims. The following detailed description of the illustrative embodiments can be understood when read in conjunction with the following drawings, where like structure is indicated with like reference numerals and in which:

FIG. 1 schematically depicts an exploded, perspective view of a fecal grinder assembly, according to one or more embodiments shown and described herein;

FIG. 2 schematically depicts a section view of the fecal grinder assembly of FIG. 1 in a grinding position, according to one or more embodiments shown and described herein;

FIG. 3 schematically depicts a section view of the fecal grinder assembly of FIG. 1 in a filtering position, according to one or more embodiments shown and described herein;

FIG. 4 schematically depicts an exploded, perspective view of a fecal syringe assembly, according to one or more embodiments shown and described herein;

FIG. 5 schematically depicts a section view of the fecal syringe assembly of FIG. 4, according to one or more embodiments shown and described herein;

FIG. 6 schematically depicts a filter member coupled to a syringe body of the fecal syringe assembly of FIG. 4, according to one or more embodiments shown and described herein;

FIG. 7 schematically depicts a section view of the filter member taken along line 7-7 of FIG. 6, according to one or more embodiments shown and described herein;

FIG. 8 schematically depicts an exploded, perspective view of a fecal mixer, according to one or more embodiments shown and described herein;

FIG. 9 schematically depicts a section view of the fecal mixer of FIG. 8, according to one or more embodiments shown and described herein;

FIG. 10 schematically depicts a perspective view of a fecal grinder assembly, according to one or more embodiments shown and described herein;

FIG. 11 schematically depicts a bottom, perspective view of a grinder of the fecal grinder assembly of FIG. 10, according to one or more embodiments shown and described herein;

FIG. 12 schematically depicts a section view of the fecal grinder assembly of FIG. 10, according to one or more embodiments shown and described herein;

FIG. 13 schematically depicts a perspective view of an automated operator for operating the fecal syringe assembly of FIG. 4, according to one or more embodiments shown and described herein; and

FIG. 14 schematically depicts a perspective view of an automated operator for operating the fecal grinder assembly of FIG. 1, according to one or more embodiments shown and described herein.

### DETAILED DESCRIPTION

Embodiments described herein are directed to fecal grinder assemblies, fecal syringe assemblies, and fecal mixers. The fecal grinder assemblies, fecal syringe assemblies, and fecal mixers are utilized to grind a fecal sample provided within an open interior volume of the various assemblies and mixers for purposes of testing and examining the fecal sample. The various assemblies and mixers generally include a container or syringe, a plunger or collection member, and a filter member. The fecal sample may be ground between abutting components such as, for example, the container and the collection member, the syringe and the plunger, or the filter member and an adjacent structure. Thereafter, the ground fecal sample is mixed with a diluent such as, for example, water, and filtered through the filter member. Various embodiments of the fecal collection apparatus and the operation of the fecal collection apparatus are described in more detail herein. Whenever possible, the same reference numerals will be used throughout the drawings to refer to the same or like parts.

Directional terms as used herein—for example up, down, right, left, front, back, top, bottom—are made only with reference to the figures as drawn and are not intended to imply absolute orientation.

Unless otherwise expressly stated, it is in no way intended that any method set forth herein be construed as requiring that its steps be performed in a specific order, nor that with any apparatus specific orientations be required. Accordingly, where a method claim does not actually recite an order to be followed by its steps, or that any apparatus claim does not actually recite an order or orientation to individual components, it is in no way intended that an order or orientation be inferred. This holds for any possible non-express basis for interpretation, for example and without limitation, matters of logic with respect to arrangement of steps, operational flow, order of components, or orientation of components; plain meaning derived from grammatical organization or punctuation, and; the number or type of embodiments described in the specification.

As used herein, the singular forms “a,” “an” and “the” include one or more referents unless the context clearly dictates otherwise. Thus, for example, reference to “a” component includes aspects having a single component or multiple components, unless the context clearly indicates otherwise.

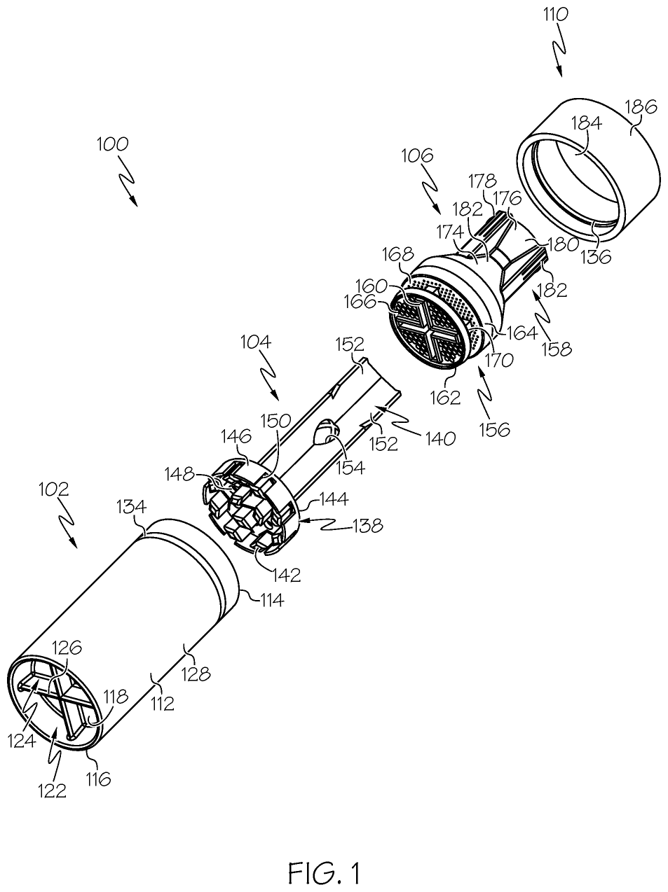

Referring now to FIG. 1, an exploded view of a fecal grinder assembly 100 in a disassembled state is illustrated, according to one or more embodiments described herein. The fecal grinder assembly 100, in embodiments, includes a container 102, a grinder 104, and a collection member 106. As described in more detail herein, the grinder 104 is at least partially received within the collection member 106. In embodiments, the grinder 104 and the collection member 106 are positionable within the container 102. In some embodiments, the fecal grinder assembly 100 includes a cap 110 securable onto the container 102 for securing the grinder 104 and the collection member 106 within the container 102.

Referring now to FIGS. 1-3, the components of the fecal grinder assembly 100 are described in more detail. The container 102 includes an outer wall 112 having a top end 114 and a bottom end 116 opposite the top end 114. The container 102 includes a bottom wall 118 extending from the outer wall 112 proximate the bottom end 116. The outer wall 112 and the bottom wall 118 of the container 102 define an open interior volume 120 in which the grinder 104 and the collection member 106 are received.

In some embodiments, the bottom wall 118 and the outer wall 112 define a recess 122 defined on a side of the bottom wall 118 opposite the open interior volume 120. A reinforcing structure 124 may be provided within the recess 122 extending from the bottom wall 118 and opposite sides of the outer wall 112 to increase the structural integrity of the container 102 as compared to containers that do not include a reinforcing structure, and to resist plastic deformation. As shown in FIG. 1, the reinforcing structure 124 includes one or more cross members 126 provided within the recess 122 and extending transverse to one another from the bottom wall 118 and opposite sides of the outer wall 112. However, it should be appreciated that other embodiments of the reinforcing structures 124 may be provided.

In embodiments, the open interior volume 120 of the container 102 has a cylindrical shape. In some embodiments, the outer wall 112 of the container 102 has a cylindrical shape and defines the shape of the open interior volume 120. It should be appreciated that the outer wall 112 of the container 102 may have an outer wall surface 128 and an inner wall surface 130 opposite the outer wall surface 128 that define the open interior volume 120. In embodiments, the outer wall surface 128 may have any suitable shape other than a cylindrical shape while maintaining a cylindrical shape of the inner wall surface 130 of the outer wall 112 and thus maintaining a cylindrical shape of the open interior volume 120.

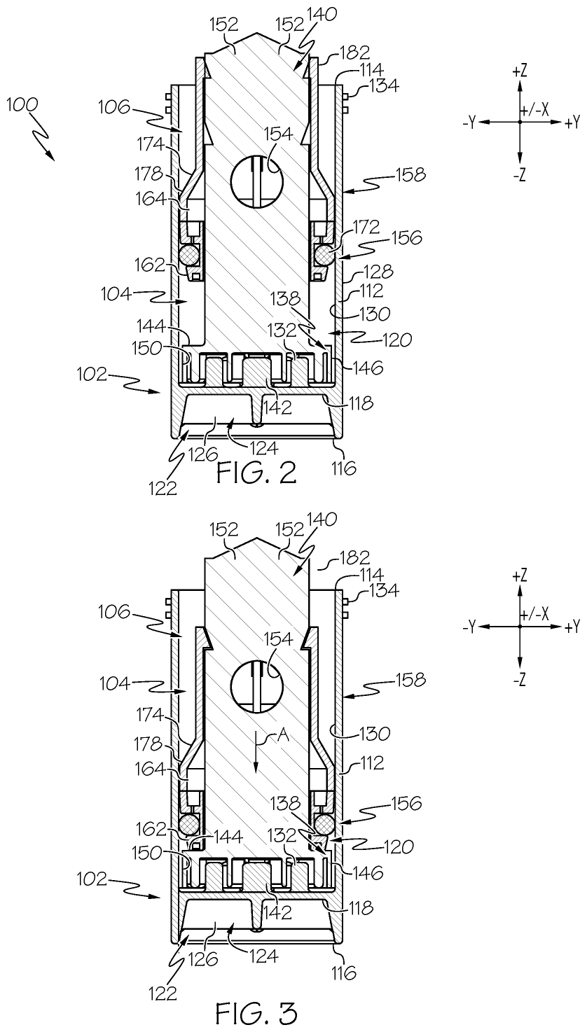

As shown in FIGS. 2 and 3, in embodiments, the container 102 includes one or more container teeth 132 extending from the bottom wall 118 of the container 102. As shown, the container 102 includes a plurality of container teeth 132 spaced apart from one another. The container teeth 132 may have any suitable shape for facilitating grinding of a fecal sample. For example, individual teeth of the container teeth 132 may be diamond-shaped having acute edges, may be rectangular prism-shaped, conical-shaped, frustoconical-shaped, or any suitable combination thereof. Moreover, while each of the container teeth 132 in the embodiment depicted in FIGS. 2 and 3 are depicted as having the same shape, it should be understood that this is merely an example, and individual teeth of the container teeth 132 can have different shapes. In some embodiments, the container teeth 132 have serrated edges.

In embodiments, the container 102 includes a locking mechanism provided proximate the top end 114 of the container 102 for securing the cap 110 (FIG. 1) to the container 102. For example, the locking mechanism may include an outer thread 134, as shown, for mating with an inner thread 136 of the cap 110. In other embodiments, the locking mechanism may include a magnet, resilient clasp, or the like.

Referring again to FIG. 1, the grinder 104 includes a base 138, a key 140 extending from the base 138, and the one or more grinder teeth 142 extending from the base 138 in a opposite the key 140. As shown, the grinder 104 includes a plurality of grinder teeth 142 spaced apart from one another. In embodiments, individual grinder teeth of the plurality of grinder teeth 142 may have any suitable shape for facilitating grinding of a fecal sample. For example, individual teeth of the plurality of grinder teeth 142 may be diamond-shaped having acute edges, may be rectangular prism-shaped, conical-shaped, frustoconical-shaped, or any suitable combination thereof. Moreover, while each of the grinder teeth 142 in the embodiment depicted in FIG. 1 are depicted as having the same shape, it should be understood that this is merely an example, and individual teeth of the grinder teeth 142 can have different shapes. In some embodiments, the grinder teeth 142 have serrated edges. As described in more detail herein, the grinder teeth 142 are spaced apart from one another to permit the grinder teeth 142 to be received within spaces provided between adjacent container teeth 132 of the container 102 such that the grinder 104 may be rotatable relative to the container 102 to allow the container teeth 132 and the grinder teeth 142 to cooperate to grind a fecal sample.

The base 138 of the grinder 104 includes a top wall 144 and a sidewall 146 extending from the top wall 144. The top wall 144 has one or more apertures 148 defined therein and extending through the top wall 144 to permit fluid to flow from one side of the top wall 144 proximate the grinder teeth 142 to an opposite side of the top wall 144 proximate the key 140. The sidewall 146 circumscribes the grinder teeth 142 and has one or more cutouts 150 defined therein. The cutouts 150 also permit fluid to flow from one side of the top wall 144 proximate the grinder teeth 142 to the opposite side of the top wall 144 proximate the key 140. In embodiments, the cutouts 150 are equidistantly spaced apart from one another about a circumference of the sidewall 146 of the base 138, however, it should be understood that this is merely an example.

In embodiments, the key 140 includes one or more key portions 152 extending from the top wall 144 of the base 138 of the grinder 104. The key 140 may include any number of key portions 152 such as, for example, one, two, or three, or more than three key portions 152. In embodiments in which the key 140 includes three or more key portions 152, the key portions 152 may be radially and equidistantly spaced apart from one another. In embodiments, a passthrough 154 may be defined, extending through the key 140. For example, in the embodiment depicted in FIG. 1, the passthrough 154 is defined at an intersection of each of the key portions 152 to permit fluid to flow across each of the key portions 152. However, it should be appreciated that one or more passthroughs 154 may be provided within each of the key portions 152 themselves rather than at the intersection of each of the key portions 152.

Referring to FIGS. 1-3, in embodiments, the collection member 106 includes a filter portion 156 and a collection body 158 extending from the filter portion 156. As described in more detail herein, a keyway 160 extends through the filter portion 156 and the collection body 158 such that the collection member 106 may engage the grinder 104. The filter portion 156 includes one or more filters for filtering a mixture of a diluent and the fecal sample. In some embodiments, the filter portion 156 includes a first filter 162 and a second filter 164 spaced apart from the first filter 162. The first filter 162, in embodiments, includes a plurality apertures 166 having a first diameter. The second filter 164, in embodiments, includes a plurality of apertures 168 having a second diameter different than the first diameter. In some embodiments, the first diameter of the apertures 166 is larger than the second diameter of the apertures 168, such that the plurality of apertures 166 filters larger particulates, while the apertures 168 filter smaller particulates than the apertures 166. As such, the first filter 162 may provide an initial filtering to filter out larger particulates and the second filter 164 may provide a second filtering to filter out smaller particulates. To position the first filter 162 and the second filter 164 in a spaced apart manner, in embodiments, the filter portion 156 includes a filter connection 170 extending between the first filter 162 and the second filter 164. In some embodiments, a sealing member 172 is provided within a space between the first filter 162 and the second filter 164. The sealing member 172, in embodiments, circumscribes the filter connection 170. The sealing member 172 may be any suitable component such as, for example, an O-ring or the like, to provide a fluid tight seal between the collection member 106 and the container 102 with the collection member 106 positioned within the open interior volume 120 of the container 102. The sealing member 172 restricts fluid from flowing around the collection member 106 bypassing the first filter 162 and the second filter 164.

The collection body 158 of the collection member 106 extends from the filter portion 156. In embodiments, the collection body 158 is coupled to the filter portion 156. In some embodiments, the collection body 158 is integrally formed as a one-piece, monolithic structure with the filter portion 156. The collection body 158 includes a first conical portion 174 extending from the filter portion 156, particularly the second filter 164, and a second conical portion 176 extending from the first conical portion 174. The first conical portion 174 includes a first sidewall 178 tapering radially inwardly toward a center of the collection member 106 in a direction opposite the filter portion 156. The second conical portion 176 includes a second sidewall 180 tapering radially inwardly toward the center of the collection member 106 in a direction toward the filter portion 156. In some embodiments, the collection body 158 includes one or more flanges 182 extending between the first sidewall 178 of the first conical portion 174 and the second sidewall 180 of the second conical portion 176. As shown, a plurality of flanges 182 extend between the first sidewall 178 of the first conical portion 174 and the second sidewall 180 of the second conical portion 176. It should be appreciated that the number of flanges 182 provided corresponds to the number of key portions 152 of the grinder 104.

As described herein, the collection member 106 defines the keyway 160 for receiving the key 140 of the grinder 104 to permit the key 140 of the grinder 104 to extend through the collection member 106. In the embodiment depicted in FIGS. 1-3, the keyway 160 extends through the first filter 162, the filter connection 170, the second filter 164, and the flanges 182. With the key 140 of the grinder 104 extending through the keyway 160 of the collection member 106, the grinder 104 and the collection member 106 engage one another to become rotatably fixed thereto such that rotation of the collection member 106 results in rotation of the grinder 104.

Referring to FIG. 1, the cap 110 is illustrated. The cap 110 includes a top wall 184 and a sidewall 186 extending from the top wall 184. The cap 110 is removably secured onto the top end 114 of the outer wall 112 of the container 102 in any suitable manner. For example, in some embodiments, the cap 110 includes an inner thread 136 that threadedly engages the outer thread 134 of the container 102, if provided. In some embodiments, the cap 110 includes a magnet, resilient clasp, or the like engaging corresponding structure on the container 102 to removably secure the cap 110 onto the container 102. In some embodiments, the cap 110 is press-fitted onto the container 102. In embodiments, the cap 110 may be removably sealed to the container 102 by being snap-fitted thereto and/or including a resilient material seal. The cap 110 may include an O-ring provided for providing a fluid tight seal with the container 102.

Referring now to FIG. 2, the fecal grinder assembly 100 is shown in an assembled state with the grinder 104 and the collection member 106 positioned within the open interior volume 120 of the container 102. In use, a fecal sample may be collected by pressing the base 138 of the grinder 104 into fecal matter such that the grinder teeth 142 engage with the fecal sample. Once the fecal sample is collected by the grinder 104, the grinder 104 may be engaged with the collection member 106 and inserted into the container 102.

As shown in FIG. 2, the grinder 104 is engaged with the container 102 such that the container teeth 132 and the grinder teeth 142 mate, i.e., are concentrically arranged, and engage with one another. The collection member 106 is positionable within the container 102 between a grinding position (FIG. 2) and a filtering position (FIG. 3). In the grinding position, as shown in FIG. 2, the collection member 106 is engaged with the grinder 104 such that the key 140 of the grinder 104 extends through the keyway 160 of the collection member 106. However, in the grinding position, the collection member 106 is positioned relative to the grinder 104 such that the first filter 162 is spaced apart from the base 138 of the grinder 104. In the grinding position, the collection member 106 may be grasped by a user and rotated, which results in simultaneous rotation of the grinder 104, with respect to the container 102, to grind the fecal sample between the container teeth 132 and the grinder teeth 142. It should be appreciated that a diluent may be positioned within the open interior volume 120 of the container 102 to mix with the fecal sample being ground by the grinder 104.

After the fecal sample is sufficiently ground, the collection member 106 may be pressed into the open interior volume 120 of the container 102 such that the first filter 162 moves in the direction of arrow A (FIG. 3) toward the base 138 of the grinder 104. Pressing the collection member 106 toward the base 138 of the grinder 104 positions the collection member 106 into the filtering position, as shown in FIG. 3. As the collection member 106 is pressed toward the base 138 of the grinder 104, the mixture of diluent and fecal sample are forced to pass through the first filter 162 and the second filter 164. Thereafter, the remaining mixture of diluent and fecal sample passes through the first conical portion 174 and the second conical portion 176. The mixture within the second conical portion 176 may be extracted, such as by using a pipette, and dispensed onto a slide for testing and examination. As described herein, the mixture of diluent and fecal sample may be dispensed onto a slide. Once on the slide, the mixture of diluent and fecal sample may be tested and examined for purposes of identifying and detecting the presence of parasites or parasite eggs and ova.

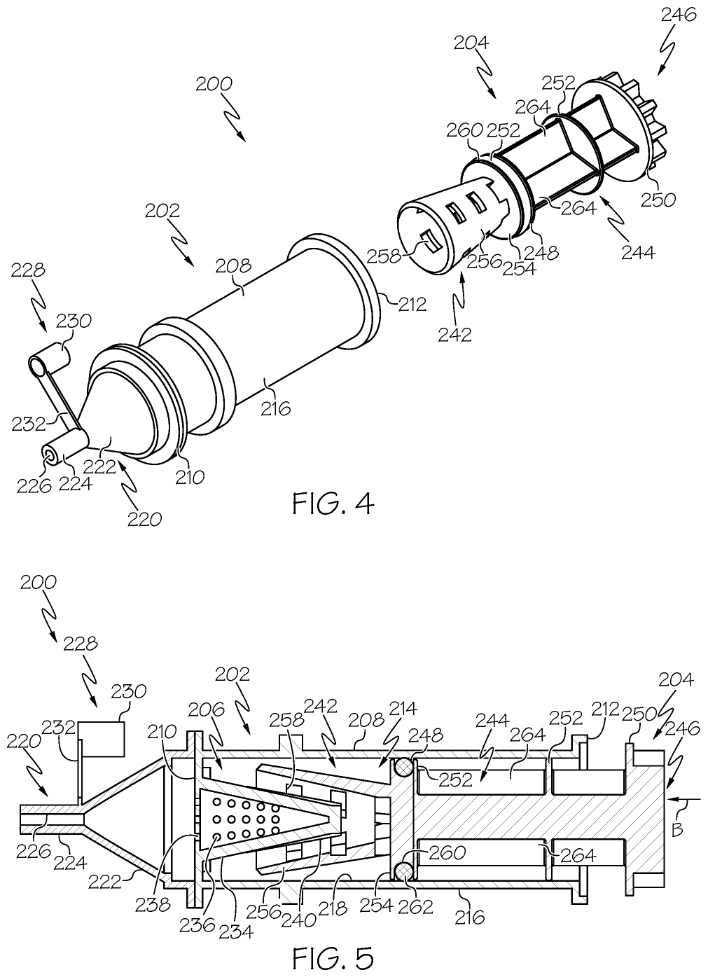

Referring now to FIGS. 4 and 5, a fecal syringe assembly 200 is illustrated. The fecal syringe assembly 200 is shown in FIG. 4 in a disassembled state and shown in FIG. 5 in an assembled state. The fecal syringe assembly 200 generally includes a syringe body 202, a plunger 204, and a filter member 206 coupled to the syringe body 202. As described in more detail herein, the plunger 204 is at least partially received within the syringe body 202.

The syringe body 202 includes an outer wall 208 including a dispensing end 210 and a receiving end 212 opposite the dispensing end 210. The outer wall 208 defines an open interior volume 214 in which the plunger 204 is received. In embodiments, the open interior volume 214 of the syringe body 202 has a cylindrical shape defined by the outer wall 208 of the syringe body 202 also having a cylindrical shape. It should be appreciated that the outer wall 208 of the syringe body 202 may have an outer wall surface 216 and an inner wall surface 218 opposite the outer wall surface 216 that defines the open interior volume 214. As such, the outer wall surface 216 may have any suitable shape other than a cylindrical shape while maintaining a cylindrical shape of the inner wall surface 218 of the outer wall 208 and thus maintaining a cylindrical shape of the open interior volume 214.

The syringe body 202 includes a nozzle 220 provided at the dispensing end 210. In embodiments, the nozzle 220 may be integrally formed with the outer wall 208 as a one-piece, monolithic structure extending from the dispensing end 210. In some embodiments, the nozzle 220 may be removably or permanently fixed to the dispensing end 210 of the outer wall 208 of the syringe body 202. In embodiments, the nozzle 220 includes a conical portion 222 provided at the dispensing end 210 of the outer wall 208 of the syringe body 202. In some embodiments, the nozzle 220 includes an elongated portion 224 extending from the conical portion 222 in a direction opposite the dispensing end 210. A channel 226 passes through the elongated portion 224 and/or the conical portion 222 such that an interior of the nozzle 220 is in fluid communication with the open interior volume 214 of the syringe body 202. In some embodiments, the nozzle 220 includes a cap 228 for closing off an open end of the elongated portion 224 of the nozzle 220. As shown in FIGS. 4 and 5, the cap 228 includes a capping member 230 and a flexible strap 232 extending from the capping member 230 to the nozzle 220. The capping member 230, in embodiments, may be removably coupled to the elongated portion in any suitable manner, such as interference-fit or the like. In embodiments including a flexible strap 232, the flexible strap 232 couples the capping member 230 to the nozzle 220 to prevent the capping member 230 from being misplaced when the nozzle 220 is uncapped. Accordingly, the flexible strap 232 and, in embodiments, the capping member 230, may be formed from a flexible material such as, for example, a deformable plastic, elastomer, or the like.

As shown in FIG. 5, in embodiments, the filter member 206 is positioned within the open interior volume 214 of the syringe body 202. More particularly, the filter member 206 may be integrally formed as a one-piece, monolithic structure with the outer wall 208 of the syringe body 202. However, as discussed in more detail herein, the filter member 206 may be removably coupled to the syringe body 202 and/or positioned at the nozzle 220 exterior to the syringe body 202. In embodiments, the filter member 206 includes a tapered sidewall 234 extending from the dispensing end 210 of the outer wall 208 of the syringe body 202. Specifically, the tapered sidewall 234 has an inner diameter that decreases as the tapered sidewall 234 extends further from the dispensing end 210 of the syringe body 202. The tapered sidewall 234 has a plurality of filter apertures 236 formed therein and extending through the filter member 206. Additionally, one or more dispensing apertures 238 are formed within the dispensing end 210 of the outer wall 208 of the syringe body 202 at a location from which the filter member 206 extends. Accordingly, as discussed in more detail herein, fluid may enter the nozzle 220 through the elongated portion 224 and pass through the dispensing apertures 238 and the filter apertures 236 to enter the open interior volume 214 of the syringe body 202. In embodiments, the filter member 206 may include one or more serrated edges 240 formed on an outer surface of the tapered sidewall 234 which facilitate breaking apart a fecal sample being ground by the plunger 204, as discussed in more detail herein.

Referring again to FIGS. 4 and 5, the plunger 204 includes a collection portion 242, a plunger body 244, and a gripping portion 246. The collection portion 242 is provided at a first end 248 of the plunger body 244 and the gripping portion 246 is provided at a second end 250 of the plunger body 244 opposite the first end 248 of the plunger body 244 and the collection portion 242. In embodiments, the collection portion 242, the plunger body 244, and the gripping portion 246 are integrally formed as a one-piece, monolithic structure. In some embodiments, the collection portion 242 and/or the gripping portion 246 coupled to the plunger body 244.

In embodiments, the plunger body 244 includes one or more outwardly extending discs 252. The discs 252 have an outer diameter substantially equal to an inner diameter of the syringe body 202. As shown, the plunger body 244 includes a pair of discs 252 with a first one of the pair of discs 252 extending proximate the first end 248 of the plunger body 244 and a second one of the pair of discs 252 extending proximate the second end 250 of the plunger body 244. The discs 252 stabilize the plunger 204 when inserted into the open interior volume 214 of the syringe body 202 by engaging the outer wall 208 of the syringe body 202.

Referring still to FIGS. 4 and 5, the collection portion 242 includes a base wall 254 and a conical sidewall 256 extending from the base wall 254. The base wall 254 is provided at the first end 248 of the plunger body 244 and the conical sidewall 256 extends from the base wall 254 in a direction opposite the first end 248 of the plunger body 244. The conical sidewall 256 has an inner diameter that increases as the conical sidewall 256 extends further from the base wall 254. Accordingly, the shape of the conical sidewall 256 is complementary with a shape of the tapered sidewall 234 of the filter member 206. The conical sidewall 256 of the collection portion 242 includes a plurality of collection apertures 258 formed therein. In some embodiments, the collection apertures 258 are equidistantly spaced apart from one another, however, it should be understood that this is merely an example. In some embodiments, a groove 260 is defined in an outer circumference of the base wall 254 of the collection portion 242 and a sealing member 262 is positioned at least partially within the groove 260. The sealing member 262 may be any suitable component such as, for example, an O-ring or the like, to provide a fluid tight seal between the plunger 204 and the syringe body 202 when the plunger 204 is positioned within the open interior volume 214 of the syringe body 202.

As discussed herein, the gripping portion 246 is positioned at the second end 250 of the plunger body 244 opposite the collection portion 242. The gripping portion 246 has an outer diameter greater than an inner diameter of the receiving end 212 of the syringe body 202. The gripping portion 246 may include a friction-inducing structure such as, for example, a plurality of ribs 264 to facilitate a user being able to grip the gripping portion 246 and rotate the plunger 204 when positioned within the open interior volume 214 of the plunger 204.

In use, the plunger 204 may be used to collect a fecal sample by pressing the collection portion 242 of the plunger 204 into fecal matter such that a fecal sample becomes lodged within the conical sidewall 256 of the collection portion 242. Once the fecal sample is collected by the plunger 204, the plunger 204 may be inserted into the syringe body 202.

As shown in FIG. 5, the plunger 204 is inserted into the open interior volume 214 of the syringe body 202 through the receiving end 212 of the syringe body 202 such that the conical sidewall 256 of the collection portion 242 at least partially surrounds the tapered sidewall 234 of the filter member 206 within the syringe body 202. The fecal sample collected within the collection portion 242 of the plunger 204 is compressed between the collection portion 242 and the filter member 206, and the plunger 204 may be rotated by grasping the gripping portion 246 of the plunger 204. Rotating the plunger 204 causes the fecal sample compressed between the collection portion 242 and the filter member 206 to break apart and particulates having a diameter smaller than a diameter of the filter apertures 236 are permitted to pass through the filter member 206. A diluent be aspirated into the nozzle 220 of the syringe body 202 and the assembled syringe body 202 and plunger 204 may be agitated back and forth to mix the diluent with the ground fecal sample. The plunger 204 may be further pressed into the syringe body 202 in the direction of arrow B to force a mixture of diluent and fecal sample to be dispensed from the nozzle 220. It should be appreciated that the mixture may be dispensed onto a slide for testing or examination to detect the presence of parasites or parasite eggs.

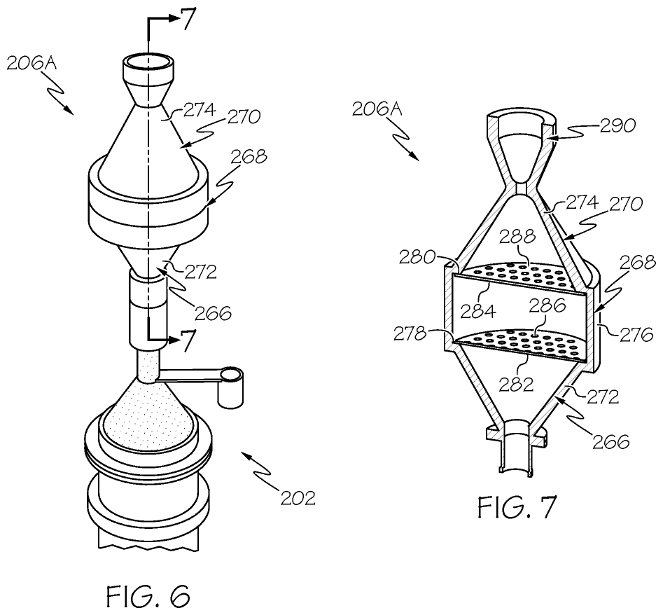

It should be appreciated that the filter member 206 may be coupled to the syringe body 202 exteriorly rather than being provided within the open interior volume 214 of the syringe body 202. As shown in FIGS. 6 and 7, in some embodiments, a filter member 206A may be coupled to the nozzle 220 of the syringe body 202 alternatively or in addition to the filter member 206 (FIG. 5). In embodiments, the filter member 206A may be coupled to the elongated portion 224 of the nozzle 220 by any suitable means such as by being press fit into the elongated portion 224, receiving an end of the elongated portion 224, or being fixed to the elongated portion 224 by a mechanical fastener, threaded engagement, leur, adhesive, or the like.

In the embodiment depicted in FIGS. 6 and 7, the filter member 206A includes a first conical portion 266, a filter portion 268, and a second conical portion 270. The filter portion 268 is positioned between the first conical portion 266 and the second conical portion 270. As shown in FIG. 7, the first conical portion 266 includes a first conical wall 272 having an inner diameter increasing as the first conical portion 266 extends toward the filter portion 268. Similarly, the second conical portion 270 includes a second conical wall 274 having an inner diameter increasing as the second conical portion 270 extends toward the filter portion 268.

The filter portion 268 includes a filter wall 276 extending between the first conical wall 272 and the second conical wall 274. In embodiments, an inner diameter of the filter wall 276 is greater than an inner diameter of the first conical wall 272 and the second conical wall 274. Thus, a first lip 278 is formed at a joining portion of the first conical wall 272 and the filter wall 276, and a second lip 280 is formed at a joining portion of the second conical wall 274 and the filter wall 276. In embodiments, the filter portion 268 includes one or more filters. For example, in the embodiment depicted in FIG. 7, the filter portion 268 includes a first filter 282 and a second filter 284 positioned within the filter portion 268 and spaced apart from one another. More particularly, the first filter 282 abuts against and is fixed to the first lip 278 and the second filter 284 abuts against and is fixed to the second lip 280. The first filter 282 and the second filter 284 may be fixed within the filter portion 268 in any suitable matter such as by a mechanical fastener, adhesive, or the like. In embodiments, the first filter 282 includes a plurality of apertures 286 having a first diameter. In some embodiments, the second filter 284 has a plurality of apertures 288. The plurality of apertures 286 of the first filter 282 and the plurality of apertures 288 of the second filter 284, in some embodiments, are different. For example, in some embodiments, the plurality of apertures 286 of the first filter 282 are larger than the plurality of apertures 288 of the second filter 284, such that the first filter 282 filters larger particulates and the second filter 284 filters smaller particulates. As such, the first filter 282 may provide an initial filtering to filter out larger particulates and the second filter 284 may provide a second filtering to filter out smaller particulates. In embodiments, the apertures 286 of the first filter 282 have a diameter of between 0.1 mm and 0.5 mm. In embodiments, the apertures 286 of the first filter 282 have a diameter of between 0.2 mm and 0.4 mm. In embodiments, the apertures 286 of the first filter 282 have a diameter of 0.3 mm. In embodiments, the apertures 288 of the second filter 284 have a diameter of between 0.6 mm and 1.4 mm. In embodiments, the apertures 288 of the second filter 284 have a diameter of between 0.8 mm and 1.2 mm. In embodiments, the apertures 288 of the second filter 284 have a diameter of 1.0 mm.

In embodiments, the filter member may include a collection tip 290 extending from the second conical portion 270 opposite the filter portion 268. The collection tip 290 may be provided to permit diluent and fecal matter to be drawn out of the filter member 206A such as by using a pipette. In embodiments, the first conical portion 266, the filter portion 268, the second conical portion 270, and the collection tip 290 are integrally formed as a one-piece, monolithic structure.

In the present embodiment in which the filter member 206A is provided exteriorly of the syringe body 202, it should be appreciated that diluent is similarly inserted into the syringe body 202 in the manner discussed herein to mix with the fecal sample. The syringe body 202 may then be agitated to mix the diluent with the fecal sample. Thereafter, the plunger 204 (FIG. 5) is inserted into the syringe body 202 to push the mixture through the filter member 206A and either into the collection tip 290 or onto a slide, filter paper, or other examination member for testing or examination to detect the presence of parasites or parasite eggs.

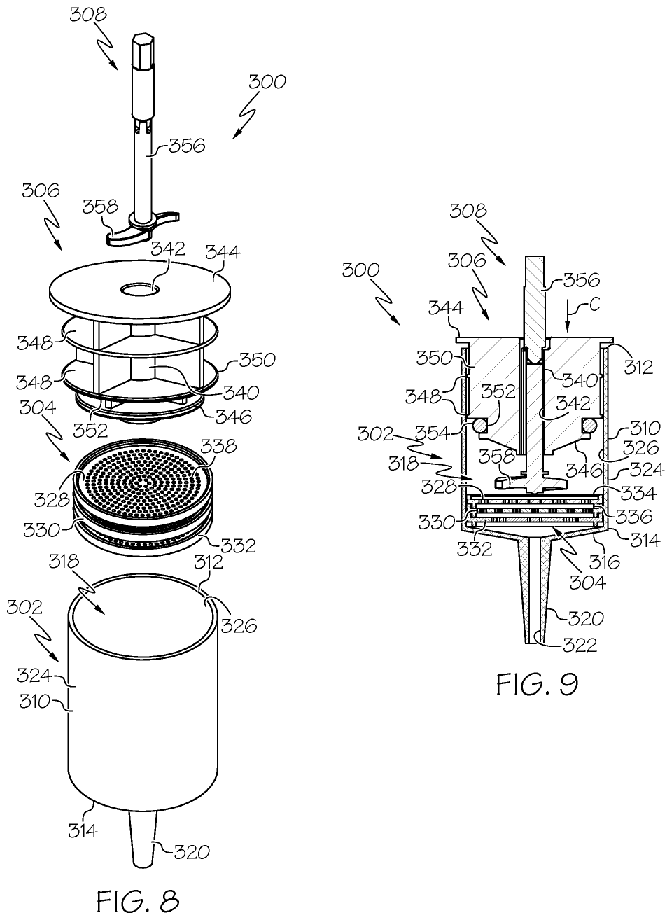

Referring now to FIGS. 8 and 9, a fecal mixer 300 is illustrated. An exploded view of the fecal mixer 300 is shown in FIG. 8 in a disassembled state and shown in FIG. 9 in an assembled state. The fecal mixer 300 generally includes a container 302, a filter member 304, a plunger 306, and a mixer 308. The container 302 includes an outer wall 310 having a receiving end 312 and a dispensing end 314 opposite the receiving end 312. The container 302 includes a bottom wall 316 extending from the outer wall 310 at the dispensing end 314. The outer wall 310 and the bottom wall 316 of the container 302 define an open interior volume 318 in which the filter member 304 and the plunger 306 are received, as described in more detail herein. The container 302, in embodiments, includes a nozzle 320 extending from the bottom wall 316 in a direction opposite the receiving end 312. As shown in FIG. 9, a channel 322 extends through the nozzle 320 placing the nozzle 320 in fluid communication with the open interior volume 318 of the container 302.

In embodiments, the open interior volume 318 of the container 302 has a cylindrical shape defined by the outer wall 310 of the container 302 also having a cylindrical shape. It should be appreciated that the outer wall 310 of the container 302 may have an outer wall surface 324 and an inner wall surface 326 opposite the outer wall surface 324 that defines the open interior volume 318. As such, the outer wall surface 324 may have any suitable shape other than a cylindrical shape while maintaining a cylindrical shape of the inner wall surface 326 of the outer wall 310 and thus maintaining a cylindrical shape of the open interior volume 318.

The filter member 304 is positionable within the open interior volume 318 of the container 302 and includes one or more filters. For example, the filter member 304 may include one filter, two filters, three filters, or more than three filters. As shown in FIGS. 8 and 9, the filter member 304 includes a first filter 328, a second filter 330, and a third filter 332 arranged in a vertical stack. As referred to herein, the first filter 328 may be located furthest from the dispensing end 314 of the container 302, the second filter 330 is provided between the first filter 328 and the third filter 332, and the third filter 332 is located closest to the dispensing end 314 of the container 302.

As shown in FIG. 9, the filters 328, 330, 332 each include a lower rim 334 and an upper rim 336 such a gap is provided between the apertures of adjacent filters 328, 330, 332. The lower rim 334 and the upper rim 336 of the filters 328, 330, 332 are radially offset from one another such that the lower rim 334 of one of the filters 328, 330, 332 does not abut against the upper rim 336 of an adjacent one of the filters 328, 330, 332.

In embodiments, each filter 328, 330, 332 defines a plurality of filter apertures 338 extending therethrough to filter particulates of a predetermined size. In embodiments, the filter apertures 338 of each of the filters 328, 330, 332 differ in diameter. For example, in the embodiment depicted in FIGS. 8 and 9, the first filter 328 includes a plurality filter apertures 338 having a first diameter for filtering larger particulates having a first diameter, the second filter 330 includes a plurality of filter apertures 338 having a second diameter less than the first diameter for filtering smaller particulates, and the third filter 332 includes a plurality of filter apertures 338 having a third diameter less than the second diameter for filtering even smaller particulates. As such, the first filter 328 may provide an initial filtering to filter out larger particulates, the second filter 330 may provide a second filtering to filter out smaller particulates, and the third filter 332 may provide a third filtering to filter out even smaller particulates.

Referring to FIG. 8, in embodiments, the plunger 306 includes a plunger body 340 having a channel 342 extending therethough, a top wall 344, a bottom wall 346 opposite the top wall 344, and one or more radially extending discs 348 provided between the top wall 344 and the bottom wall 346. The top wall 344 has an outer diameter greater than an inner diameter of the outer wall 310 of the container 302. The discs 348 have an outer diameter substantially equal to an inner diameter of the outer wall 310 of the container 302. As shown, the plunger body 340 includes a pair of discs 348. The discs 348 stabilize the plunger 306 when inserted into the open interior volume 318 of the container 302 by abutting against the outer wall 310 of the container 302 without significant space therebetween. In embodiments, reinforcing members 350 extend between the top wall 344 and the bottom wall 346, as well as the discs 348. The reinforcing members 350, in embodiments, restrict deformation of the discs 340 upon the application of force. The bottom wall 346 has an outer diameter less than the inner diameter of the outer wall 310 of the container 302. In embodiments, a groove 352 is defined between the bottom wall 346 and an adjacent one of the discs 348 in which a sealing member 354 may be provided, as shown in FIG. 9. The sealing member 354 may be any suitable component such as, for example, an O-ring or the like, to provide a fluid tight seal between the plunger 306 and the container 302 with the plunger 306 positioned within the open interior volume 318 of the container 302.

As shown in FIG. 8, the mixer 308 includes a shaft 356 and a mixing member 358 fixed to an end of the shaft 356. In some embodiments, the shaft 356 can be coupled to a motor (not shown), which rotates the shaft 356 and the mixing member 358. In some embodiments, the shaft 356 can be rotated manually by a user. The shaft 356 of the mixer 308 is positionable to extend through the channel 342 of the plunger body 340.

Referring now to FIG. 9, the fecal mixer 300 is shown in the assembled state with the filter member 304 provided within the open interior volume 318 of the container 302 proximate the nozzle 320, the plunger 306 is inserted into the open interior volume 318 of the container 302, and the mixer 308 is positioned such that the shaft 356 extends through the channel 342 formed in the plunger body 340 of the plunger 306 and the mixing member 358 is positioned between the plunger 306 and the filter member 304. Accordingly, a fecal sample may be mixed with diluent within the open interior volume 318 of the container 302 by operating the mixer 308. Once mixed, the plunger 306 may be inserted further into the container 302 in the direction of arrow C to force the mixture of diluent and fecal sample through the filter member 304 and be dispensed out of the container 302 through the nozzle 320 such as onto a slide for testing or examination to detect the presence of parasites or parasite eggs.

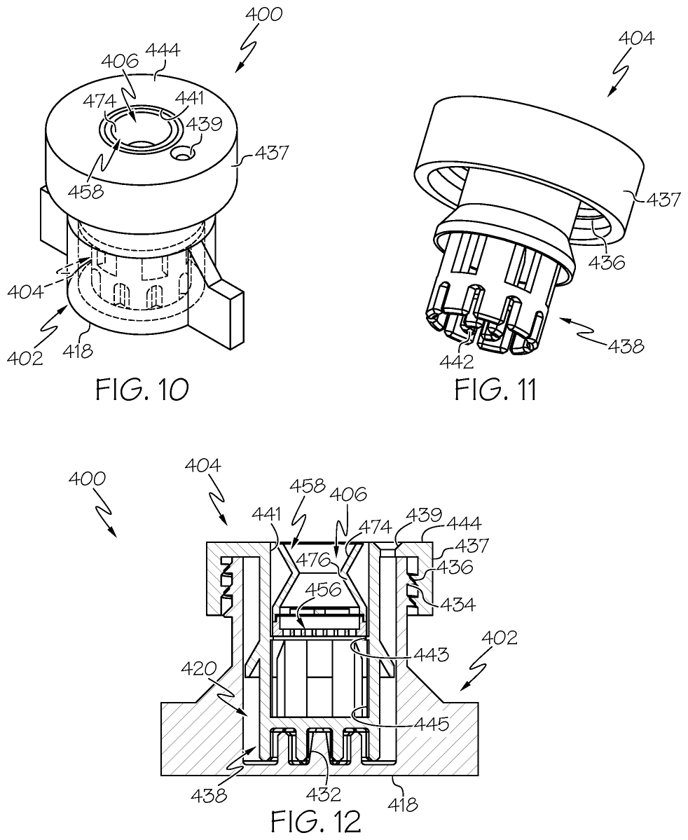

Referring now to FIGS. 10-12, another fecal grinder assembly 400 generally includes a container 402, a grinder 404, and a collection member 406. It should be appreciated that the fecal grinder assembly 400 is substantially similar to the fecal grinder assembly 100 described herein and illustrated in FIGS. 1-3. Specifically, the container 402 defines an open interior volume 420 and a plurality of container teeth 432 extending from a bottom wall 418 of the container 402. Similarly, the grinder 404 includes a plurality of grinder teeth 442 extending from a base 438 of the grinder 404 that cooperate with the container teeth 432 as the grinder 404 is inserted into the open interior volume 420 of the container 402, as shown in FIG. 12. In some embodiments, the grinder 404 includes inner threads 436 extending from a flange 437 of the grinder 404 that threadedly engage outer threads 434 of the container 402 to retain the grinder 404 within the open interior volume 420 of the container 402. In some embodiments, an aperture 439 is defined in a top wall 444 of the grinder 404 from which the flange 437 extends. A diluent may be inserted through the aperture 439 to enter the open interior volume 420 of the container 402 to mix with a fecal sample collected by the grinder 404 in the manner discussed herein with respect to the fecal grinder assembly 100 of FIGS. 1-3.

The fecal grinder assembly 400 differs from the fecal grinder assembly 100 of FIGS. 1-3 as the collection member 406, which includes a filter portion 456 and a collection body 458, is received within a recess 441 defined in the grinder 404. The collection member 406 abuts against a lip 443 formed on an inner wall 445 of the grinder 404 and may be fixed thereto by any suitable means, such as an adhesive or the like. As with the fecal grinder assembly 100 of FIGS. 1-3, the collection member 406 of the fecal grinder assembly 400 includes a first conical portion 474 and a second conical portion 476 extending from the filter portion 456.

In use, with the fecal grinder assembly 400 in the assembled state, as shown in FIG. 12, diluent is inserted into the open interior volume 420 of the container 402 through the aperture 439 formed in the top wall 444 of the grinder 404, and the grinder 404 is rotated to grind the fecal sample between the container teeth 432 and the grinder teeth 442. After a period of time, particulates, such as eggs and ova from the fecal sample float toward the top of the diluent, are filtered through the filter portion 456, and settle within the second conical portion 476 of the collection member 406. From there, the particulates may be extracted, such as by using a pipette, and dispensed onto a slide for testing and examination to detect the presence of parasites or parasite eggs.

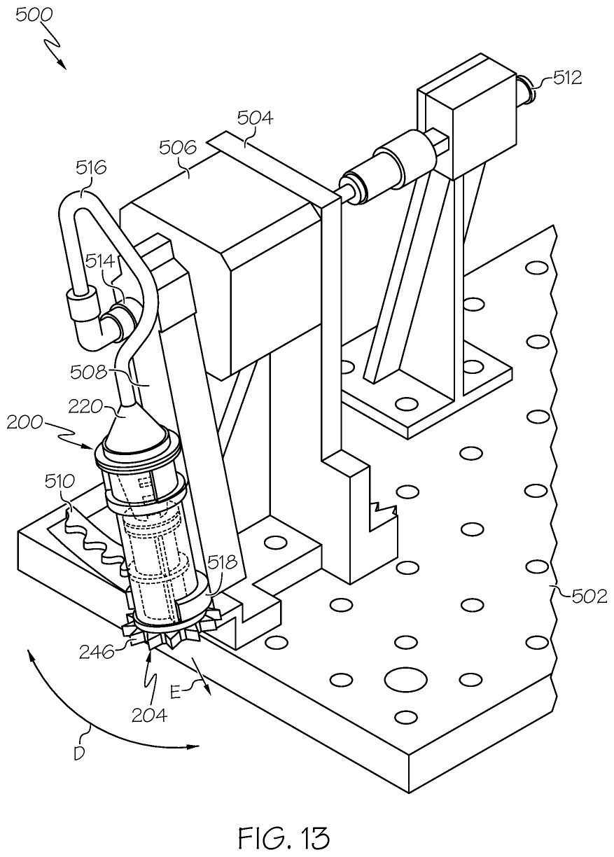

Referring now to FIG. 13, an automated operator 500 is illustrated for aspirating and mixing the contents within the fecal syringe assembly 200 depicted in FIGS. 4 and 5. The automated operator 500 includes a platform 502, a mounting bracket 504 mounted atop the platform 502, a motor 506 mounted to the mounting bracket 504, and a fixing arm 508 rotatably coupled to the motor 506. The mounting bracket 504 includes an arcuate lower toothed member 510 provided proximate a lower end of the mounting bracket 504. The automated operator 500 includes a fluid coupling 512 for delivering fluid to the fecal syringe assembly 200. The fluid coupling 512 may be provided on the mounting bracket 504 or a separate mounting structure provided on the platform 502. Although not shown, a channel extends from the fluid coupling 512, through the motor 506 and to an outlet port 514 on the fixing arm 508. A conduit 516 may be provided to extend from the outlet port 514 of the fixing arm 508 to the nozzle 220 of the fecal syringe assembly 200, which may be fixed to the fixing arm 508 by one or more retaining clips 518. When the fecal syringe assembly 200 is fixed to the fixing arm 508, the fecal syringe assembly 200 is positioned such that the gripping portion 246 of the plunger 204 engages the arcuate lower toothed member 510.

In use, the motor 506 is operated to rotate the fixing arm 508 in alternating clockwise and counterclockwise directions, as shown by arrow D. Rotating the fixing arm 508 results in the gripping portion 246 of the plunger 204 being rotated due to engagement with the arcuate lower toothed member 510 and thus grinding of the fecal sample in the manner discussed herein. After a predetermined period of time in which it is determined that the fecal sample is sufficiently ground, fluid, such as a diluent, may be introduced into the fluid coupling 512 and into the fecal syringe assembly 200 through the conduit 516 and the nozzle 220, which pushes the plunger 204 in the direction of arrow E to cause the gripping portion 246 to disengage the arcuate lower toothed member 510. Thereafter, the motor 506 may be operated once again to rotate the fixing arm 508 in alternating clockwise and counterclockwise directions, as shown by arrow D, to mix the fecal sample with the fluid. Operation of the motor 506 may be increased to increase a rotational speed of the fecal syringe assembly 200 to cause a centrifugal effect. This causes particulates within the mixture to float to the top of the fecal syringe assembly 200 and may be subsequently extracted in the manner discussed herein, such as by pipetting.

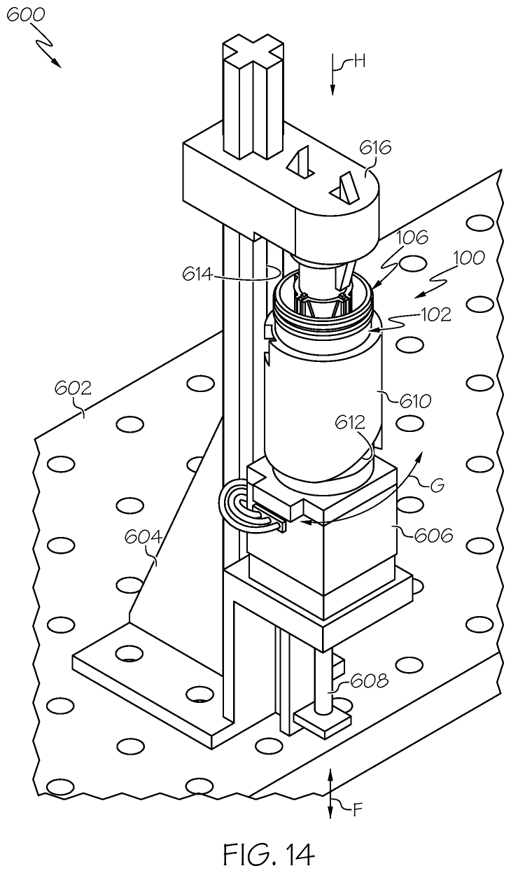

Referring now to FIG. 14, an automated operator 600 is illustrated for mixing the contents of the fecal grinder assembly 100 depicted in FIGS. 1-3. The automated operator 600 includes a platform 602, a mounting bracket 604 mounted atop the platform 602, a motor 606 mounted to the mounting bracket 604, and a lead screw 608 extending through the motor 606. As shown, a cup 610 is provided on the motor 606 in which the fecal grinder assembly 100 is positioned. The lead screw 608 extends through and is threadedly engaged with the cup 610. The cup 610 includes a helical groove 612 formed in an outer surface thereof that mates with a key 614 formed in the mounting bracket 604. The container 102 of the fecal grinder assembly 100 is fixed within the cup 610 and the collection member 106, positioned within the container 102, is fixed to a movable arm 616 extending from the mounting bracket 604.

In use, the motor 606 is operated to move the lead screw 608 in alternating vertical directions in the direction of arrow F. Due to the threaded engagement between the lead screw 608 and the cup 610, and the helical groove 612 of the cup 610 engaging the key 614 formed in the mounting bracket 604, vertical translation of the lead screw 608 in the direction of arrow F results in rotation of the cup 610 in the direction of arrow G. Accordingly, rotation of the cup 610 in the direction of arrow G results in similar rotation of the container 102 of the fecal grinder assembly 100, which is fixed to the cup 610. With the collection member 106 rotatably fixed relative to the container 102 by the movable arm 616, a fecal sample within the container 102 may be ground and mixed with a diluent provided therein. After a predetermined amount of time, the movable arm 616 may be actuated, such as by a separate motor (not shown), to lower the collection member 106 toward in a direction of arrow H toward the motor 606 and filter the mixture of fecal sample and diluent in the manner discussed herein to detect the presence of parasites or parasite eggs.

From the above, it is to be appreciated that defined herein are fecal grinder assemblies, fecal syringe assemblies, and fecal mixers. The fecal grinder assemblies, fecal syringe assemblies, and fecal mixers, are utilized to grind a fecal sample provided within an open interior volume of the various assemblies and mixers and mix the ground fecal sample with a diluent for purposes of testing and examining the fecal sample. The various assemblies and mixers generally include a container or syringe, a plunger or collection member, and a filter member. The fecal sample may be ground between abutting components such as, for example, the container and the collection member, the syringe and the plunger, or the filter member and an adjacent structure. Thereafter, the ground fecal sample is mixed with the diluent and filtered through the filter member. It will be apparent to those skilled in the art that various modifications and variations can be made to the embodiments described herein without departing from the scope of the claimed subject matter. Thus, it is intended that the specification cover the modifications and variations of the various embodiments described herein provided such modification and variations come within the scope of the appended claims and their equivalents.

What is claimed is:

1. A fecal grinder assembly comprising:
a container defining an open interior volume and a plurality of container teeth extending from a bottom wall of the container and within the open interior volume;
a grinder slidably positioned within the open interior volume of the container, the grinder comprising a base and a key extending from the base in a first direction and a plurality of grinder teeth extending from the base in a second direction opposite the first direction; and
a collection member comprising a filter portion and a collection body extending from the filter portion, a keyway extends through the filter portion and the collection body for receiving the key of the grinder when the collection member is positioned on the grinder and within the open interior volume of the container.

2. The fecal grinder assembly of claim 1, wherein the base of the grinder comprises a top wall and a sidewall extending from the top wall, the sidewall circumscribing the plurality of grinder teeth, wherein a plurality of cutouts are formed in the sidewall.

3. The fecal grinder assembly of claim 2, wherein the plurality of cutouts are equidistantly spaced apart from one another along the sidewall of the grinder.

4. The fecal grinder assembly of claim 2, wherein the key comprises a plurality of key portions extending from the top wall of the base of the grinder.

5. The fecal grinder assembly of claim 2, wherein the top wall of the base of the grinder comprises a plurality of apertures formed therein and extending through the top wall.

6. The fecal grinder assembly of claim 1, wherein rotation of the collection member when the key of the grinder is received within the keyway of the collection member causes the grinder to rotate within the open interior volume of the container.

7. The fecal grinder assembly of claim 1, wherein the collection body of the collection member comprises a first conical portion extending from the filter portion, and a second conical portion extending from the first conical portion.

8. The fecal grinder assembly of claim 7, wherein the first conical portion comprises a first sidewall tapering toward a center of the collection member in a direction opposite the filter portion.

9. The fecal grinder assembly of claim 7, further comprising a sealing member provided within a groove formed in the filter portion.

10. A fecal syringe assembly comprising:
a syringe body comprising a dispensing end, a receiving end opposite the dispensing end, and a nozzle formed at the dispensing end;
a plunger partially insertable into the syringe body through the receiving end, the plunger comprising a collection portion and a gripping portion opposite the collection portion, the gripping portion comprising an outer diameter greater than an inner diameter of the receiving end of the syringe body; and
a filter member provided at one of the nozzle or within the syringe body proximate the collection portion of the plunger when the plunger is inserted into the receiving end of the syringe body.

11. The fecal syringe assembly of claim 10, wherein the filter member is fixed within the interior of the syringe body.

12. The fecal syringe assembly of claim 11, wherein the filter member includes a tapered sidewall having a plurality of apertures formed therein and extending through the filter member.

13. The fecal syringe assembly of claim 12, wherein the collection portion of the plunger comprises a conical sidewall corresponding to a shape of the tapered sidewall of the filter member, the conical sidewall of the collection portion of the plunger comprising a plurality of apertures formed therein and extending through the conical sidewall.

14. The fecal syringe assembly of claim 10, further comprising a sealing member provided within a groove formed between the collection portion and the gripping portion.

15. The fecal syringe assembly of claim 10, wherein the filter member is coupled to the nozzle of the syringe body.

16. The fecal syringe assembly of claim 15, wherein the filter member comprises:
a first conical portion proximate the nozzle;
a filter portion extending from the first conical portion, a pair of spaced apart filters provided within the filter portion; and
a second conical portion extending from the filter portion and opposite the first conical portion.

17. A fecal mixer comprising:
a container comprising a dispensing end, a receiving end opposite the dispensing end, and a nozzle formed at the dispensing end;
a filter member positioned within the container proximate the dispensing end;
a plunger insertable into the container through the receiving end, the plunger comprising a sealing member encircling a lower wall portion of the plunger; and
a mixer comprising a shaft extending through the plunger and a mixing member provided at a first end of the shaft, the mixing member positioned between the filter member and the plunger when the plunger is inserted into the container.

18. The fecal mixer of claim 17, wherein the filter member comprises a first filter and a second filter stacked on the first filter.

19. The fecal mixer of claim 18, wherein the first filter comprises a plurality of apertures each having a first diameter and the second filter comprises a plurality of apertures each having a second diameter greater than the first diameter, the first filter positioned between the second filter and the dispensing end of the container.

20. The fecal mixer of claim 18, wherein the first filter and the second filter each include a lower rim and an upper rim radially offset from one another to define a gap between a center of the first filter and a center of the second filter.
